(19) United States
(12) Patent Application Publication
(10) Pub. No.: US 2010/0292436 A1
(43) Pub. Date: Nov. 18, 2010

(54) METHOD FOR PRODUCING BIVALIRUDIN

(75) Inventors: Juncai BAI, Shanghai (CN); Ruoping ZHANG, Shanghai (CN); Yadong LIU, Shanghai (CN); Guoqing ZHANG, Shanghai (CN)

Correspondence Address: MATTHIAS SCHOLL, 14781 MEMORIAL DRIVE, SUITE 1319, HOUSTON, TX, 77079, US

(73) Assignee: SHANGHAI AMBIOPHARM, INC., Shanghai (CN)

(21) Appl. No.: 12/781,037

(22) Filed: May 17, 2010

(30) Foreign Application Priority Data
May 15, 2009 (CN) 200910051311.X

(51) Int. Cl. C07K 14/00; C07D 211/00

(52) U.S. Cl. 530/326; 546/184

(57) ABSTRACT

A method for producing bivalirudin using solid phase peptide synthesis by the following steps: a) mixing a Fmoc-amino acid resin or a Fmoc-peptide resin with a de-protective agent so as to remove Fmoc-; b) in the presence of a condensing agent, condensing a Fmoc- or Boc-amino acid with the amino acid or the peptide bound to the resin; c) repeating the steps a) and b) to yield a peptide resin represented by Formula I,

(SEQ ID NO. 1)
Boc-D-Phe¹-Pro²-Arg(Pbf)³-Pro⁴-Gly⁵-Gly⁶-Gly⁷-
Gly⁸-Asn(Trt)⁹-Gly¹⁰-Asp(OtBu)¹¹-Phe¹²-Glu
(OtBu)¹³-Glu(OtBu)¹⁴-Ile¹⁵-Pro¹⁶-Glu(OtBu)¹⁷-Glu
(OtBu)¹⁸-Tyr(tBu)¹⁹-Leu²⁰-Resin (I)

and d) in the presence of a cleavage agent, separating the peptide from the resin to yield bivalirudin represented by Formula II (SEQ ID NO. 2).

D-Phe-Pro-Arg-Pro-Gly-Gly-Gly-Gly-Asn-Gly-Asp-Phe-
Glu-Glu-Ile-Pro-Glu-Glu-Tyr-Leu (II)

Based on its total volume, the de-protective agent is composed of between 3 and 20% of piperidine and between 0.5 and 10% of bicyclic amidine. The method is low in cost and the resultant bivalirudin has high purity.

### CROSS-REFERENCE TO RELATED APPLICATIONS

Pursuant to 35 U.S.C. §119 and the Paris Convention Treaty, this application claims the benefit of Chinese Patent Application No. 200910051311.X filed May 15, 2009, the contents of which are incorporated herein by reference.

### BACKGROUND OF THE INVENTION

1. Field of the Invention

The invention relates to a method for producing a peptide, and more particularly to a method for producing bivalirudin using solid phase peptide synthesis technique.

2. Description of the Related Art

Solid phase peptide synthesis technique, a breakthrough for producing peptide, is firstly invented by R. Bruce Merrifield. That is, linking a first amino acid whose amino group is protected by a protecting group to a solid phase support, removing the protecting group with a de-protective agent, activating a carboxyl of a protected second amino acid with N,N′-dicyclohexyl carbodiimide (DCC), and reacting the first amino acid with the second amino acid to yield a protected dipeptide at the solid phase support. Repeat the above steps, which makes the peptide chain grow from C-terminal to N-terminal. After a required chain length is obtained, the protecting group is removed, and the ester bond between the peptide chain and the solid phase carrier is hydrolyzed with strong acid HF. Thus, a peptide is obtained. The synthesis technique is actually a process of adding amino acid repetitively, and the synthesis order is from the C-terminal (carboxyl terminal) to the N-terminal (amino terminal).

Thrombin inhibitors are considered as a promising anti-thrombosis drug. Bivalirudin, an anticoagulant peptide, is a bivalent hirudin (hirulog). Hirudin is a peptide therapeutically effective for inhibiting thrombin and extracted from a blood-sucking leech, i.e., Hirudo medicinalis, with 20 amino acids.

US20070093423A discloses a method for producing bivalirudin using solid phase peptide synthesis technique. Actually, it is a combination of solid phase and liquid phase synthesis method which is very difficult for practice. In addition, the cleavage agent used therein includes acids, ethanedithiol, etc. Thus, the method has high cost, and the resultant product has many impurities.

Thus, it is very urgent to design a method for producing bivalirudin using solid phase peptide synthesis technique that has low cost and by which the resultant bivalirudin has high purity, particularly the purities of the glycine-deletion and glycine addition closed eluted with the main peak of HPLC can be reduced to less than 0.10% after the preparative HPLC purification to meet the pharmaceutical requirements.

### SUMMARY OF THE INVENTION

In view of the above-described problems, it is one objective of the invention to provide a method for producing bivalirudin using solid phase peptide synthesis.

It is another objective of the invention to provide a de-protective agent for solid phase peptide synthesis.

It is still another objective of the invention to provide a use of a cleavage agent for solid phase peptide synthesis.

It is still another objective of the invention to provide a use of pentafluorophenol for solid phase peptide synthesis.

To achieve the above objectives, in accordance with one embodiment of the invention, there is provided a method for producing bivalirudin using solid phase peptide synthesis, the method comprising
a) mixing a Fmoc-amino acid resin or a Fmoc-peptide resin with a de-protective agent so as to remove Fmoc-;
b) in the presence of a condensing agent, condensing a Fmoc- or Boc-amino acid with the amino acid or the peptide bound to the resin;
c) repeating the steps a) and b) to yield a peptide resin represented by Formula I (SEQ ID NO. 1),

Boc-D-Phe¹-Pro²-Arg(Pbf)³-Pro⁴-Gly⁵-Gly⁶-Gly⁷-
Gly⁸-Asn(Trt)⁹-Gly¹⁰-Asp(OtBu)¹¹-Phe¹²-Glu
(OtBu)¹³-Glu(OtBu)¹⁴-Ile¹⁵-Pro¹⁶-Glu(OtBu)¹⁷-Glu
(OtBu)¹⁸-Tyr(tBu)¹⁹-Leu²⁰-Resin (I)
and

d) in the presence of a cleavage agent, separating the peptide from the resin to yield bivalirudin represented by Formula II (SEQ ID NO. 2).

D-Phe-Pro-Arg-Pro-Gly-Gly-Gly-Gly-Asn-Gly-Asp-Phe-
Glu-Glu-Ile-Pro-Glu-Glu-Tyr-Leu (II)

In a class of this embodiment, prior to the step a), mixing a resin or a resin linked to an amino acid or a peptide with a protected amino acid to yield the Fmoc-amino acid resin or the Fmoc-peptide resin. Particularly, the resin is a Wang resin.

In a class of this embodiment, based on its total volume, the de-protective agent comprises between 3 and 20% of piperidine and between 0.5 and 10% of bicyclic amidine (DBU).

In a class of this embodiment, the de-protective agent further comprises between 0 and 20% of 1-hydroxy benzotriazole (HOBt), between 0 and 8% of 3-hydroxy-1,2,3-benzo triazine-4(3H)-one (HOOBt), or a mixture thereof.

In a class of this embodiment, based on its total volume, the de-protective agent comprises between 5 and 15% of piperidine and between 1 and 7% of bicyclic amidine (DBU).

In a class of this embodiment, the de-protective agent further comprises between 0.5 and 10% of 1-hydroxy benzotriazole (HOBt), between 2 and 5% of 3-hydroxy-1,2,3-benzo triazine-4(3H)-one (HOOBt), or a mixture thereof.

In a class of this embodiment, in the steps a) and b), the amino acid bound to the resin is Leucine; in the step b), the Boc-amino acid is Boc-D-Phe-OH.

In a class of this embodiment, in the step b), Fmoc-Arg(Pbf)-OH, pentafluorophenol, and the condensing agent are mixed so as to prompt the condensation of Fmoc-Arg(Pbf)-OH with the peptide bound to the resin.

In a class of this embodiment, the condensing agent is N,N′-diisopropyl carbodiimide (DIC), O-(7-aza-benzotriazole-1-yl)-N,N,N′,N′-tetramethyl uronium hexafluoro phosphate (HATU), O-(benzotriazole-1-yl)-N,N,N,N-4-methyl-uronium tetrafluoroborate (TBTU)/N-methyl morpholine (NMM), (benzo triazol-1-yl-O)tripyrrolidine phosphonium hexafluorophosphate (PyBOP), 1-hydroxy benzotriazole (HOBt), or a mixture thereof.

In a class of this embodiment, the cleavage agent comprises trifluoroacetic acid (TFA), triisopropyl silane (TIS), and water, with a volume ratio thereof 95-60:5-10:5-30.

In a class of this embodiment, the cleavage agent is cooled to 0±2° C. by an ice-water bath or a refrigerant.

In a class of this embodiment, the step b) is repeated for between 1 and 3 times.

In accordance with another embodiment of the invention, there is provided a de-protective agent for solid phase peptide synthesis, wherein the de-protective agent, based on its total volume, comprises between 3 and 20% of piperidine and between 0.5 and 10% of bicyclic amidine (DBU).

In a class of this embodiment, the de-protective agent further comprises between 0 and 20% of 1-hydroxy benzotriazole (HOBt), between 0 and 10% of 3-hydroxy-1,2,3-benzo triazine-4(3H)-one (HOOBt), or a mixture thereof.

In a class of this embodiment, based on its total volume, the de-protective agent comprises between 5 and 15% of piperidine and between 1 and 7% of bicyclic amidine (DBU).

In a class of this embodiment, the de-protective agent further comprises between 0.5 and 10% of 1-hydroxy benzotriazole (HOBt), between 0.2 and 5% of 3-hydroxy-1,2,3-benzo triazine-4(3H)-one (HOOBt), or a mixture thereof.

In accordance with still another embodiment of the invention, there is provided a method for producing bivalirudin using solid phase peptide synthesis comprising applying a de-protective agent, wherein the de-protective agent, based on its total volume, comprises between 3 and 20% of piperidine and between 0.5 and 10% of bicyclic amidine (DBU), and the peptide comprises the structure of -Asn-Gly-.

In a class of this embodiment, the de-protective agent further comprises between 0 and 20% of 1-hydroxy benzotriazole (HOBt), between 0 and 10% of 3-hydroxy-1,2,3-benzo triazine-4(3H)-one (HOOBt), or a mixture thereof.

In accordance with still another embodiment of the invention, there is provided a method for producing bivalirudin using solid phase peptide synthesis comprising applying pentafluorophenol to condense Fmoc-Arg(Pbf)-OH with an amino acid or peptide bound to a resin.

In a class of this embodiment, the method comprises the step of mixing 1.5-6.0 equivalents of Fmoc-Arg(Pbf)-OH, pentafluorophenol, 1.5-6.0 equivalents of condensing agent, and a resin linked to an amino acid or a peptide for between 12 and 36 hrs.

In a class of this embodiment, the condensing agent is N,N′-diisopropyl carbodiimide (DIC), O-(7-aza-benzotriazole-1-yl)-N,N,N′,N′-tetramethyl uronium hexafluoro phosphate (HATU), O-(benzotriazole-1-yl)-N,N,N,N-4-methyl-uronium tetrafluoroborate (TBTU), (benzo triazol-1-yl-O)tripyrrolidine phosphonium hexafluorophosphate (PyBOP), 1-hydroxy benzotriazole (HOBt), N-methyl morpholine (NMM), or a mixture thereof.

Thus, the invention provides a method for producing bivalirudin using solid phase peptide synthesis technique that has low cost and by which the resultant bivalirudin has high purity, particularly the glycine-deletion and glycine-addition closely eluted with the main peak of HPLC can be reduced to less than 0.10% after the preparative HPLC purification to meet the pharmaceutical impurity requirements.

Advantages of the invention are summarized below:
1) Low cost: compared with conventional methods for producing bivalirudin, the method reduces cost by about 50%;
2) Low impurities and high purity: the purity of the crude bivalirudin of the invention resulting from this synthesis process is 77-93%, and the glycine-deletion and glycine-addition impurities closely eluted with the main peak can be controlled less than 0.10% after the preparative HPLC purification to meet the pharmaceutical requirements;
3) Low risk factor: methyl tert-butyl ether (MTBE) instead of ether is used in the invention, which improves the safety of production. Ether is an extremely flammable chemical with flash point of −45° C., and boiling point of 34.6° C. Methyl tert-butyl ether has a flash point of −28° C. and a boiling point of 55.3° C.; and
4) environment-friendly: the method is a solid phase synthesis process, almost no water involved in, and the organic solvents for washing can be recycled and thereby waste is very little.

### DETAILED DESCRIPTION OF THE EMBODIMENTS

Studies show that during producing bivalirudin using solid phase peptide synthesis technique, if a de-protective agent comprising DMF, piperidine, DBU, HOBt, HOOBT, or a mixture thereof is used, the impurities can be minimized. Particularly, DBU has a very important influence on the purities prior to the main peak.

Further studies show that during producing bivalirudin using solid phase peptide synthesis technique, for the structure of -Asn-Gly-, a de-protective agent comprising piperidine, DBU, HOBt, HOOBT, or a mixture thereof is particularly effective.

In addition, studies show that during producing bivalirudin using solid phase peptide synthesis technique, upon condensation, a condensing agent comprising HOBt/DIC or TBTU/NMM can be added and the whole condensation process can be monitored. For steps of condensing Arginine (Arg), pentafluorophenol is required so as to reduce the production cost and reduce the Arginine deletion impurity.

The meaning of the abbreviations of the invention is listed as follows:

| | |
|---|---|
| Fmoc | 9-fluorenylmethoxycarbonyl |
| Boc | Butoxycarbonyl |
| DMF | N,N-dimethylformamide |
| KSCN | Potassium thiocyanate |
| DBU | 1,8-diazabicyclo(5.4.0)undec-7-ene |
| HOBt | 1-hydroxy benzotriazole |
| DIC | N,N′-diisopropyl c + arbodiimide |
| NMM | N-methyl morpholine |
| Pbf | 2,2,4,6,7-5-pentamethyl-benzofuran-5-sulfonyl |
| Opfp | Pentafluorophenyl ester |
| TFA | Trifluoroacetic acid |
| TIS | Triisopropyl silane |
| MTBE | Methyl tert-butyl ether |
| HOOBT | 3-hydroxy-1,2,3-benzo triazine-4(3H)-one |
| HATU | O-(7-aza-benzotriazole-1-yl)-N,N,N′,N′- |
| | tetramethyl uranium hexafluoro phosphate |
| TBTU | O-(benzotriazole-1-yl)-N,N,N,N-4-methyl-uronium |
| | tetrafluoroborate |
| PyBOP | (benzo triazol-1-yl-O)tripyrrolidine phosphonium |
| | hexafluorophosphate |

In embodiments of the invention, the de-protective agent is a chemical agent which can remove a protecting group of amino group. The protecting group of amino group is well-known to those of ordinary skill in the art and includes but is not limited to Fmoc and Boc. Particularly, based on its total volume, the de-protective agent comprises between 3 and 20% of piperidine and between 0.5 and 10% of bicyclic amidine (DBU). More particularly, the de-protective agent further comprises between 0 and 20% of 1-hydroxy benzotriazole (HOBt), between 0 and 10% of 3-hydroxy-1,2,3-benzo triazine-4(3H)-one (HOOBt), or a mixture thereof.

In embodiments of the invention, the condensing agent is a chemical agent which can prompt the formation of a peptide bond between an amino group of an amino acid and a carboxyl of another amino acid. The condensing agent is well-known to those of ordinary skill in the art and includes but is not limited to carbodiimide, ByPOB, HATU, and TBTU.

In embodiments of the invention, the cleavage agent is a chemical agent which can separate a peptide bound to a resin from the resin. The cleavage agent is well-known to those of ordinary skill in the art and includes but is not limited to a weak acid solution comprising TFA and HCl solution.

In embodiments of the invention, a method for producing bivalirudin using solid phase peptide synthesis comprises
a) loading Fmoc-Leu-OH to a resin; the resin is well-known to those of ordinary skill in the art and particularly a Wang resin, and more particularly a Wang resin having a substitution rate of 0.40-1.4 mmol/g;
b) removing Fmoc- with a de-protective agent, i.e., washing the Fmoc-Leu-resin of step a) with the de-protective agent so as to remove Fmoc-;
c) condensing a Fmoc-amino acid with the amino acid bound to the resin; in a preferable embodiment, condensing amino acid on the resin from C-terminal to N-terminal until a peptide represented by Formula II is obtained; and
d) in the presence of a cutting agent, separating the peptide represented by Formula II from the resin to yield bivalirudin represented by Formula II. The cleavage agent comprises TFA, TIS, and water.

Preferably, in step a), 1.8-3.0 resin equivalent of Fmoc-Leu-OH is reacted with a Wang resin.

Preferably, in step c), 1.5-4.5 resin equivalent of Fmoc-amino acid and 1.5-3.0 resin equivalent of HOBt are dissolved with DMF (1 mL/g resin); the mixture is added to the resin, and then 2.0-6.0 resin equivalent of DIC or TBTU is added, and allowed to react for 90 min. The resultant solution is diluted with DMF at 10° C. to a volume (4 mL/g resin) and then allowed for reaction for 6 hrs.

Preferably, in step c), the condensation of Fmoc-Arg(Pbf)-OH is as follows: 1.5-6.0 equivalents of Fmoc-Arg(Pbf)-OH and pentafluorophenol are dissolved with DMF or KSCN (3 mL/g resin), and then 1.5-6.0 equivalents of a condensing agent such as DIC, HATU, TBTU, or PyBOP are added and stirred for 90 min. The resultant Fmoc-Arg(Pbf)-OPfp/DMF solution is added to the resin and stirred for 12-36 hrs.

In embodiments of the invention, the obtained crude bivalirudin has a yield of 90-125% and purity of 77-93%. Preferably, the obtained peptide represented by Formula II is mixed with MTBE or ether to yield a peptide precipitate. More preferably, the MTBE or ether is cooled to −10 to 0° C. by an ice-water bath or a refrigerant known to those of ordinary skill in the art, and the precipitate is washed with the other ether and separated by filtration or centrifugation. The purity of the resultant bivalirudin can reach 90% or more.

The above mentioned technical features can be combined freely upon implementation.

For further illustrating the invention, experiments detailing a method for producing bivalirudin using solid phase peptide synthesis are described below. It should be noted that the following examples are intended to describe and not to limit the invention.

Unless otherwise specified, the experiments in Examples are carried out at normal conditions or in accordance with the conditions recommended by the manufacturer, and all percentage, ratio, or proportion is calculated by weight.

The volume percentage of weight of the invention is well-known to those of ordinary skill in the art, e.g., the weight of solute dissolved in 100 mL of solution.

Unless otherwise specified, the meaning of scientific terms in the invention is the same as that known to those of ordinary skill in the art. Methods or materials similar to or equal to those of the invention are practical.

The parameters of HPLC of embodiments of the invention are listed below:

| | | UV detection | |
|---|---|---|---|
| Column | C18 5 u 100 A 250 × 4.5 mm | wavelength | 215 nm |
| Mobile | A: 0.1% TFA aqueous solution | Velocity of | |
| phase | B: 0.1% TFA acetonitrile solution | flow | 1.0 mL/min |
| Test | | | |
| temper- | | Injection | |
| ature | 45° C. | volume | 5-50 μL |

Gradient:

| Time (min.) | % A | % B |
|---|---|---|
| 0.0 | 85 | 15 |
| 35.0 | 60 | 45 |
| 35.1 | 20 | 80 |
| 40.0 | 20 | 80 |
| 40.1 | 85 | 15 |
| 45.0 | 85 | 15 |

The retention time of bivalirudin is about 23.7 min and that of main impurity is between 23 and 23.3 min.

### Example 1

### Preparation of Bivalirudin I

Step 1: loading Fmoc-Leu-OH to a Resin

2.0 molar equivalents of Fmoc-Leu-OH was activated with 2,6-dichlorobenzoylchloride and pyridine and reacted with Wang resin (having a substitution rate of 0.40-1.4 mmol/g) in a DMF solution.

Step 2

Removing Fmoc: Another DMF solution comprising 15% of piperidine/5% of DBU was added and allowed to react for 30 min so as to remove Fmoc. The resultant resin was washed once with DMF, twice with methanol, and twice with DMF, respectively.

Condensing Fmoc-amino acid (the last was Boc-D-Phe-OH): 1.5-3.0 resin equivalent of Fmoc-amino acid and 1.5 resin equivalent of HOBt were dissolved with DMF (3 mL/g resin); the mixture was added to the resin, and then 3.0 resin equivalent of DIC was added, and allowed to react for 90 min. The whole process was monitored by ninhydrin colorimetric method (Kaiser). The resultant solution was diluted with DMF at 10° C. to a volume (4 mL/g resin) and then allowed for reaction for 6 hrs.

Condensing Fmoc-Arg(Pbf)-OH: 1.5 equivalents of Fmoc-Arg(Pbf)-OH and pentafluorophenol were dissolved with DMF (1 mL/g resin), and then 6.0 equivalents of HATU were added and stirred for 90 min. The resultant Fmoc-Arg(Pbf)-OPfp/DMF solution was added to the resin and stirred for 48 hrs at between 5 and 8° C. The whole process was monitored by ninhydrin colorimetric method (Kaiser).

Washing: After all required amino acids were condensed, the resin was washed twice with methanol, thrice with DMF, and thrice with methanol, respectively. Subsequently, the resin was dried under vacuum to reach a certain weight and packed, and a yield thereof was calculated according to its weight gain.

Step 3

Preparation of a cleavage agent: TFA, TIS, and water with a volume ratio of 80:10:10 were mixed in a vessel to yield a cleavage agent. The cleavage agent was cooled to 0±2° C. by an ice-water bath or a refrigerant.

Cleavage: The peptide resin was slowly added to the cooled cleavage agent. The mixture was stirred for 2-3 hrs at less than 5° C. and then was filtered. The resultant filtrate was collected.

Precipitating: To MTBE cooled to −10° C., the filtrate was added and a peptide precipitate was produced. The precipitate was washed thrice with cooled ether. Upon washing, the cooled ether should be sufficient enough to cover the precipitate in the centrifuge tube or in the filter, and the precipitate and the cooled ether were mixed completely by a spatula. The mixture was centrifugated to yield bivalirudin I.

Drying: The solid peptide of bivalirudin I (as shown in Formula II) was transferred to a vessel and dried in a vacuum drying oven or in a dryer at room temperature for more than 6 hrs. Subsequently, the solid peptide was weighed and packed.

The product had purity of 85%. The impurity (retention time of 23.2 min) prior to main peak was less than 1% in content, and the largest single impurity (retention time of 26.3 min) was less than 2.5% in content.

### Example 2

### Preparation of Bivalirudin II

Step 1: Binding Fmoc-Leu to a Resin

3.0 molar equivalents of Fmoc-Leu were activated with 2,6-dichlorobenzoylchloride and pyridine and reacted with Wang resin (having a substitution rate of 1.0-1.2 mmol/g) in a DMF solution. The unreacted groups of the resin were blocked by benzoyl chloride/triethylamine.

Step 2

Removing Fmoc: 5 times resin bed volume of DMF solution comprising 10% of piperidine/7% of DBU/3% of HOOBt was added and allowed to react for 30 min so as to remove Fmoc. The resultant resin was washed once with 5 times resin bed volume of DMF, thrice with 5 times resin bed volume of methanol, and thrice with 5 times resin bed volume of DMF, respectively.

Condensing Fmoc-amino acid (the last was Boc-D-Phe-OH): 1.5-2.0 resin equivalent of Fmoc-amino acid and 3.0 resin equivalent of HOBt were dissolved with DMF (5 mL/g resin); the mixture was added to the resin, and then 3.0 resin equivalent of TBTU/NMM was added, and allowed to react for 90 min. The whole process was monitored by ninhydrin colorimetric method (Kaiser).

Condensing Fmoc-Arg(Pbf)-OH: 6.0 equivalents of Fmoc-Arg(Pbf)-OH and pentafluorophenol were dissolved with DMF (4 mL/g resin), and then 1.5 equivalents of DIC were added and stirred for 90 min. The resultant Fmoc-Arg(Pbf)-OPfp/DMF solution was added to the resin and stirred for 18 hrs.

Washing: After all required amino acids were condensed, the resin was washed twice with 5 times resin bed volume of methanol, thrice with 5 times resin bed volume of DMF, and thrice with 5 times resin bed volume of methanol, respectively. Subsequently, the resin was dried under vacuum to reach a certain weight and packed, and a yield thereof was calculated according to its weight gain.

Step 3

Preparation of a cutting agent: TFA, TIS, and water with a volume ratio of 90:5:5 were mixed in a vessel to yield a cutting agent. The cutting agent was cooled to 0±2° C. by an ice-water bath or a refrigerant. The peptide resin was mixed with the cooled cutting agent. The mixture was allowed to react for 2-3 hrs at less than 5° C. and then was filtered. The resultant filtrate was collected.

Precipitating: To ether cooled to −10° C., the filtrate was added and a peptide precipitate was produced. The precipitate was collected by filtration or centrifugation and washed thrice with cooled MTBE. Upon washing, the cooled MTBE should be sufficient enough to cover the precipitate in the centrifuge tube or in the filter, and the precipitate and the cooled MTBE were mixed completely by a spatula. The mixture was centrifugated to yield bivalirudin II.

Drying: The solid peptide of bivalirudin II (as shown in Formula II) was transferred to a vessel and dried in a vacuum drying oven or a dryer at room temperature for more than 6 hrs. Subsequently, the solid peptide was weighed and packed.

The product had purity of 87%. The impurity (retention time of 23.5 min) prior to main peak was 0.63% in content, and the largest single impurity (retention time of 27.1 min) was 2.1% in content.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for producing bivalirudin using solid phase peptide synthesis, comprising:
a) mixing a Fmoc-amino acid resin or a Fmoc-peptide resin with a de-protective agent so as to remove Fmoc-;
b) in the presence of a condensing agent, condensing a Fmoc- or Boc-amino acid with said amino acid or said peptide bound to said resin;
c) repeating the steps a) and b) to yield a peptide resin represented by Formula I,
(SEQ ID NO. 1)
Boc-D-Phe¹-Pro²-Arg(Pbf)³-Pro⁴-Gly⁵-Gly⁶-Gly⁷-
Gly⁸-Asn(Trt)⁹-Gly¹⁰-Asp(OtBu)¹¹-Phe¹²-Glu
(OtBu)¹³-Glu(OtBu)¹⁴-Ile¹⁵-Pro¹⁶-Glu(OtBu)¹⁷-Glu
(OtBu)¹⁸-Tyr(tBu)¹⁹-Leu²⁰-Resin (I)
and
d) in the presence of a cleavage agent, separating said peptide from said resin to yield bivalirudin represented by Formula II.
(SEQ ID NO. 2)
D-Phe-Pro-Arg-Pro-Gly-Gly-Gly-Gly-Asn-Gly-Asp-Phe-
Glu-Glu-Ile-Pro-Glu-Glu-Tyr-Leu (II)

2. The method of claim 1, wherein based on its total volume, said de-protective agent comprises between 3 and 20% of piperidine and between 0.5 and 10% of bicyclic amidine.

3. The method of claim 2, wherein said de-protective agent further comprises between 0 and 20% of 1-hydroxy benzotriazole, between 0 and 8% of 3-hydroxy-1,2,3-benzo triazine-4(3H)-one, or a mixture thereof.

4. The method of claim 2, wherein based on its total volume, said de-protective agent comprises between 5 and 15% of piperidine and between 1 and 7% of bicyclic amidine.

5. The method of claim 4, wherein said de-protective agent further comprises between 0.5 and 10% of 1-hydroxy benzotriazole, between 2 and 5% of 3-hydroxy-1,2,3-benzo triazine-4(3H)-one, or a mixture thereof.

6. The method of claim 1, wherein in the steps a) and b), said amino acid bound to said resin is Leucine; in the step b), said Boc-amino acid is Boc-D-Phe-OH.

7. The method of claim 1, wherein in the step b), Fmoc-Arg(Pbf)-OH, pentafluorophenol, and said condensing agent are mixed so as to prompt the condensation of Fmoc-Arg(Pbf)-OH with said peptide bound to said resin.

8. The method of claim 1, wherein said condensing agent is N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate/N-methyl morpholine, (benzo triazol-1-yl-O)tripyrrolidine phosphonium hexafluorophosphate, 1-hydroxy benzotriazole, or a mixture thereof.

9. The method of claim 1, wherein said cutting agent comprises trifluoroacetic acid, triisopropyl silane, and water, with a volume ratio thereof 95-60:5-10:5-30.

10. A de-protective agent for solid phase peptide synthesis, wherein said de-protective agent, based on its total volume, comprises between 3 and 20% of piperidine and between 0.5 and 10% of bicyclic amidine.

11. The de-protective agent of claim 10, further comprising between 0 and 20% of 1-hydroxy benzotriazole, between 0 and 10% of 3-hydroxy-1,2,3-benzo triazine-4(3H)-one, or a mixture thereof.

12. A method for producing bivalirudin using solid phase peptide synthesis comprising applying a de-protective agent, wherein said de-protective agent, based on its total volume, comprises between 3 and 20% of piperidine and between 0.5 and 10% of bicyclic amidine, and said peptide comprises the structure of -Asn-Gly-.

13. The method of claim 12, wherein said de-protective agent further comprises between 0 and 20% of 1-hydroxy benzotriazole, between 0 and 10% of 3-hydroxy-1,2,3-benzo triazine-4(3H)-one, or a mixture thereof.

14. A method for producing bivalirudin using solid phase peptide synthesis comprising applying pentafluorophenol to condense Fmoc-Arg(Pbf)-OH with an amino acid or peptide bound to a resin.

15. The method of claim 14, comprising mixing 1.5-6.0 equivalents of Fmoc-Arg(Pbf)-OH, pentafluorophenol, 1.5-6.0 equivalents of condensing agent, and a resin linked to an amino acid or a peptide for between 12 and 36 hrs.

16. The method of claim 15, wherein said condensing agent is N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate, (benzo triazol-1-yl-O)tripyrrolidine phosphonium hexafluorophosphate, 1-hydroxy benzotriazole, N-methyl morpholine, or a mixture thereof.


SEQUENCE LISTING (2 sequences)

SEQ ID NO: 1 - protein, 20 aa, Artificial Sequence
  Fully synthetic peptide
FPRPGGGGNGDFEEIPEEYL

SEQ ID NO: 2 - protein, 20 aa, Artificial Sequence
  Fully synthetic peptide
FPRPGGGGNGDFEEIPEEYL